SEQUENCE LISTING (17 sequences)

SEQ ID NO: 1 - DNA, 567 bp, Homo sapiens
  CDS (1)..(567)
  mat_peptide (64)..(564)
atgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtga

SEQ ID NO: 2 - protein, 188 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 3 - DNA, 624 bp, Homo sapiens
  CDS (1)..(621)
  mat_peptide (64)..(621)
atgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggcttag

SEQ ID NO: 4 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 5 - DNA, 408 bp, Homo sapiens
  CDS (1)..(408)
  mat_peptide (64)..(408)
atgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagac

SEQ ID NO: 6 - protein, 136 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPD

SEQ ID NO: 7 - DNA, 5614 bp, Artificial Sequence
  Description of Artificial Sequence: pSecTagA-VEGF-B167-H6
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagtccagtgtggtggaattcggcttcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggcatcatcatcatcatcattgagcggccgctcgagtctagagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 5614 bp, Artificial Sequence
  Description of Artificial Sequence: pSecTagA-VEGF-B167-H6-NXT
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagtccagtgtggtggaattcggcttcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcacaacgtcaccatgcagatcctcatgatccggtacccgagcagtcagctgggggagatgtccctggaagaacacagccagtgtgaatgcagacctaaaaaaaaggacagtgctgtgaagccagacagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggcatcatcatcatcatcattgagcggccgctcgagtctagagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 5695 bp, Artificial Sequence
  Description of Artificial Sequence: pSecTagA-VEGF-B186-H6-NXT
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagtccagtgtggtggaattcggcttcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcacaacgtcaccatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggctcatcatcatcatcatcattgaattctgcagatatccagcacagtggcggccgctcgagtctagagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 5695 bp, Artificial Sequence
  Description of Artificial Sequence: pSecTagA-VEGF-B186-H6
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagtccagtgtggtggaattcggcttcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggctcatcatcatcatcatcattgaattctgcagatatccagcacagtggcggccgctcgagtctagagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 11 - DNA, 5458 bp, Artificial Sequence
  Description of Artificial Sequence: pSecTagA-VEGF-BEx1-5-H6
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagtccagtgtggtggaattcggcttcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagaccatcatcatcatcaccactgagcggccgctcgagtctagagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 12 - DNA, 5458 bp, Artificial Sequence
  Description of Artificial Sequence: pSecTagA-VEGF-BEx1-5-H6-NXT
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagtccagtgtggtggaattcggcttcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcacaacgtcaccatgcagatcctcatgatccggtacccgagcagtcagctgggggagatgtccctggaagaacacagccagtgtgaatgcagacctaaaaaaaaggacagtgctgtgaagccagaccatcatcatcatcaccactgagcggccgctcgagtctagagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 53 bp, Artificial Sequence
  Description of Artificial Sequence: PCR primer to introduce a N-glycosylation site at positions 289-297 of SEQ ID NO:3 (VEGF-B186)
tcggtaccggatcatgaggatctgcatggtgacgttgtgctgcccagtggcca

SEQ ID NO: 14 - DNA, 21 bp, Artificial Sequence
  Description of Artificial Sequence: PCR 5′ primer for amplification of nucleotides 250 to 567 from Genebank Acc. No. U48801
cctgacgatggcctggagtgt

SEQ ID NO: 15 - DNA, 50 bp, Artificial Sequence
  Description of Artificial Sequence:  PCR 3′ primer for amplification of nucleotides 250 to 567 from Genebank Acc. No. U48801
gagcggccgctcaatgatgatgatgatgatgccttcgcagcttccggcac

SEQ ID NO: 16 - DNA, 20 bp, Artificial Sequence
  Description of Artificial Sequence:  PCR 5′ primer for amplification of nucleotides 1 to 411 from Genebank Acc. No. U48801
caccatgagccctctgctcc

SEQ ID NO: 17 - DNA, 47 bp, Artificial Sequence
  Description of Artificial Sequence:   PCR 3′ primer for amplification of nucleotides 1 to 411 from Genebank Acc. No. U48801
gagcggccgctcagtggtgatgatgatggtctggcttcacagcactg

(19) United States
(12) Patent Application Publication
(10) Pub. No.: US 2002/0068694 A1
(43) Pub. Date: Jun. 6, 2002

(54) Glycosylated VEGF-B and method for increasing the amount of soluble VEGF-B

(76) Inventors: Markku Michael Jeltsch, Helsinki (FI); Kari Alitalo, Helsinki (FI); Birgitta Olofsson, San Francisco, CA (US); Ulf Eriksson, Stockholm (SE)

Correspondence Address: CROWELL & MORING LLP, INTELLECTUAL PROPERTY GROUP, P.O. BOX 14300, WASHINGTON, DC, 20044-4300, US

(21) Appl. No.: 09/912,436

(22) Filed: Jul. 26, 2001

Related U.S. Application Data
(60) Provisional application No. 60/220,824, filed on Jul. 26, 2000.

(51) Int. Cl. A61K 38/18; A61K 31/727; C07H 21/04; C12N 5/06; C12P 21/02

(52) U.S. Cl. 514/8; 514/56; 435/69.1; 435/325; 435/320.1; 536/23.5; 530/395

(57) ABSTRACT

N-glycosylated VEGF-B proteins, nucleic molecule encoding these proteins, pharmaceutical compositions containing them and a method for increasing the amount of a soluble VEGF-B protein. The VEGF-B proteins are useful in stimulating and maintaining angiogenesis.

### CROSS-REFERENCE TO RELATED APPLICATIONS

This application claims priority from U.S. Provisional Patent Application No. 60/220,824, filed Jul. 26, 2000.

### BACKGROUND OF THE INVENTION

This invention relates to the discovery that N-glycosylation of VEGF-B causes an increase in soluble proteins.

The two major components of the mammalian vascular system are endothelial cells and smooth muscle cells. The endothelial cells form the lining of the inner surface of all blood vessels and lymphatic vessels in the mammal. The formation of new blood vessels can occur by two different processes, vasculogenesis or angiogenesis (for a review see Risau, W., Nature 386:671-674 (1997)). Vasculogenesis is characterized by the in situ differentiation of endothelial cell precursors to mature endothelial cells and association of these cells to form vessels, such as occurs in the formation of the primary vascular plexus in the early embryo. In contrast, angiogenesis, the formation of blood vessels by growth and branching of pre-existing vessels, is important in later embryogenesis and is responsible for most of the blood vessel growth which occurs in the adult. Angiogenesis is a physiologically complex process involving proliferation of endothelial cells, degradation of extracellular matrix, branching of vessels and subsequent cell adhesion events. In the adult, angiogenesis is tightly controlled and limited under normal circumstances to the female reproductive system. However angiogenesis can be switched on in response to tissue damage. Also solid tumors are able to induce angiogenesis in surrounding tissue, thus sustaining tumor growth and facilitating the formation of metastases (Folkman, J., Nature Med. 1:27-31, (1995)). The molecular mechanisms underlying the complex angiogenic processes are far from being understood.

Angiogenesis is also involved in a number of pathological conditions, where it plays a role or is involved directly in different sequelae of the disease. Some examples include neovascularization associated with various liver diseases, neovascular sequelae of diabetes, neovascular sequelae to hypertension, neovascularization in post trauma, neovascularization due to head trauma, neovascularization in chronic liver infection (e.g. chronic hepatitis), neovascularization due to heat or cold trauma, dysfunction related to excess of hormone, creation of hemangiomas and restenosis following angioplasty. In arthritis, new capillaries invade the joint and destroy cartilage. In diabetes, new capillaries in the retina invade the vitreous humour, causing bleeding and blindness (Folkman, J. and Shing, Y., J. Biol. Chem. 267:10931-10934 (1992)). The role of angiogenic factors in these and other diseases has not yet been clearly established.

Because of the crucial role of angiogenesis in so many physiological and pathological processes, factors involved in the control of angiogenesis have been intensively investigated. A number of growth factors have been shown to be involved in the regulation of angiogenesis. These include fibroblast growth factors (FGFs), platelet-derived growth factors (PDGFs), transforming growth factor alpha (TGF), and hepatocyte growth factor (HGF). See for example Folkman et al, J. Biol. Chem., 267:10931-10934 (1992) for a review.

It has been suggested that a particular family of endothelial cell-specific growth factors known as the vascular endothelial growth factors (VEGFs) and their corresponding receptors are primarily responsible for stimulation of endothelial cell growth and differentiation, and for certain functions of the differentiated cells. These factors are members of the PDGF/VEGF family, and appear to act primarily via endothelial receptor tyrosine kinases (RTKs). The PDGF/VEGF family of growth factors belongs to the cystine-knot superfamily of growth factors, which also includes the neurotrophins and transforming growth factor-β.

Eight different proteins have been identified in the PDGF/VEGF family, namely two PDGFs (A and B), VEGF and five members that are closely related to VEGF. The five members closely related to VEGF are: VEGF-B, described in International Patent Application No. WO 96/26736 and in U.S. Pat. Nos. 5,840,693 and 5,607,918 by Ludwig Institute for Cancer Research and The University of Helsinki; VEGF-C or VEGF2, described in Joukov et al, EMBO J. 15:290-298 (1996), Lee et al, Proc. Natl. Acad. Sci. USA 93:1988-1992 (1996), and U.S. Pat. Nos. 5,932,540 and 5,935,540 by Human Genome Sciences, Inc; VEGF-D, described in International Patent Application No. PCT/US97/14696 (WO 98/07832), and Achen et al, Proc. Natl. Acad. Sci. USA 95:548-553 (1998); the placenta growth factor (PlGF), described in Maglione et al, Proc. Natl. Acad. Sci. USA 88:9267-9271 (1991); and VEGF3, described in International Patent Application No. PCT/US95/07283 (WO 96/39421) by Human Genome Sciences, Inc. Each VEGF family member has between 30% and 45% amino acid sequence identity with VEGF in their VEGF homology domain (VHD). This VEGF homology domain contains the eight conserved cysteine residues which form the cystine-knot motif. In their active, physiological state, the proteins are dimers. Functional characteristics of the VEGF family include varying degrees of mitogenicity for endothelial cells and related cell types, induction of vascular permeability and angiogenic and lymphangiogenic properties.

Vascular endothelial growth factor (VEGF) is a homodimeric glycoprotein that has been isolated from several sources. VEGF shows highly specific mitogenic activity for endothelial cells. VEGF has important regulatory functions in the formation of new blood vessels during embryonic vasculogenesis and in angiogenesis during adult life (Carmeliet et al., Nature, 380: 435-439, (1996); Ferrara et al., Nature, 380: 439-442, (1996); reviewed in Ferrara and Davis-Smyth, Endocrine Rev., 18: 4-25, (1997)). The significance of the role played by VEGF has been demonstrated in studies showing that inactivation of a single VEGF allele results in embryonic lethality due to failed development of the vasculature (Carmeliet et al., Nature, 380: 435-439, (1996); Ferrara et al., Nature, 380: 439-442, (1996)). The isolation and properties of VEGF have been reviewed; see Ferrara et al., J. Cellular Biochem., 47: 211-218, (1991) and Connolly, J. Cellular Biochem., 47: 219-223, (1991).

In addition, VEGF has strong chemoattractant activity towards monocytes, can induce the plasminogen activator and the plasminogen activator inhibitor in endothelial cells, and can also induce microvascular permeability. Because of the latter activity, it is sometimes referred to as vascular permeability factor (VPF). VEGF is also chemotactic for certain hematopoetic cells. Recent literature indicates that VEGF blocks maturation of dendritic cells and thereby reduces the effectiveness of the immune response to tumors (many tumors secrete VEGF) (Gabrilovich et al., Blood 92: 4150-4166, (1998); Gabrilovich et al., Clinical Cancer Research 5: 2963-2970, (1999)).

Vascular endothelial growth factor B (VEGF-B) has similar angiogenic and other properties to those of VEGF, but is distributed and expressed in tissues differently from VEGF. In particular, VEGF-B is very strongly expressed in heart, and only weakly in lung, whereas the reverse is the case for VEGF (olofsson, B. et al, Proc. Natl. Acad. Sci. USA 93:2576-2581 (1996)). RT-PCR assays have demonstrated the presence of VEGF-B mRNA in melanoma, normal skin, and muscle. This suggests that VEGF and VEGF-B, despite the fact that they are co-expressed in many tissues, may have functional differences. A comparison of the PDGF/VEGF family of growth factors reveals that the 167 amino acid isoform of VEGF-B is the only family member that is completely devoid of any glycosylation. Gene targeting studies have shown that VEGF-B deficiency results in mild cardiac phenotype, and impaired coronary vasculature (Bellomo et al, Circ. Res. 86:E29-35 (2000)).

Human VEGF-B was isolated using a yeast co-hybrid interaction trap screening technique by screening for cellular proteins which might interact with cellular retinoic acid-binding protein type I (CRABP-I). The isolation and characteristics including nucleotide and amino acid sequences for both the human and mouse VEGF-B are described in detail in International Application No. WO 96/26736 and in U.S. Pat. Nos. 5,840,693 and 5,607,918 by Ludwig Institute for Cancer Research and The University of Helsinki and in Olofsson et al, Proc. Natl. Acad. Sci. USA 93:2576-2581 (1996). The nucleotide sequence for human VEGF-B is also found at GenBank Accession No. U48801. The entire disclosures of WO 96/26736, U.S. Pat. No. 5,840,693 and 5,607,918 are incorporated herein by reference. The mouse and human genes for VEGF-B are almost identical, and both span about 4 kb of DNA. The genes are composed of seven exons and their exon-intron organization resembles that of the VEGF and PlGF genes (Grimmond et al, Genome Res. 6:124-131 (1996); Olofsson et al, J. Biol. Chem. 271:19310-19317 (1996); Townson et al, Biochem. Biophys. Res. Commun. 220:922-928 (1996)).

VEGF-C was isolated from conditioned media of the PC-3 prostate adenocarcinoma cell line (CRL1435) by screening for ability of the medium to induce tyrosine phosphorylation of the endothelial cell-specific receptor tyrosine kinase VEGFR-3 (Flt4), using cells transfected to express VEGFR-3. VEGF-C was purified using affinity chromatography with recombinant VEGFR-3, and was cloned from a PC-3 cDNA library. Its isolation and characteristics are described in detail in Joukov et al., EMBO J., 15: 290-298, (1996).

VEGF-D was isolated from a human breast cDNA library, commercially available from Clontech, by screening with an expressed sequence tag obtained from a human cDNA library designated “Soares Breast 3NbHBst” as a hybridization probe (Achen et al, Proc. Natl. Acad. Sci. USA, 95: 548-553, (1998)). Its isolation and characteristics are described in detail in International Patent Application No. W098/07832 and in U.S. Pat. No. 6,235,713. These documents also describe the isolation of a biologically active fragment of VEGF-D which consists of VEGF-D amino acid residues 93 to 201. The VEGF-D gene is broadly expressed in the adult human, but is certainly not ubiquitously expressed. VEGF-D is strongly expressed in heart, lung and skeletal muscle. Intermediate levels of VEGF-D are expressed in spleen, ovary, small intestine and colon, and a lower expression occurs in kidney, pancreas, thymus, prostate and testis. No VEGF-D mRNA was detected in RNA from brain, placenta, liver or peripheral blood leukocytes.

PlGF was isolated from a term placenta cDNA library. Its isolation and characteristics are described in detail in Maglione et al., Proc. Natl. Acad. Sci. USA, 88: 9267-9271, (1991). Presently its biological function is not well understood.

VEGF3 was isolated from a cDNA library derived from colon tissue. VEGF3 is stated to have about 36% identity and 66% similarity to VEGF. The method of isolation of the gene encoding VEGF3 is unclear and no characterization of the biological activity is disclosed in International Patent Application No. PCT/US95/07283 (WO 96/39421).

Similarity between two proteins is determined by comparing the amino acid sequence and conserved amino acid substitutions of one of the proteins to the sequence of the second protein, whereas identity is determined without including the conserved amino acid substitutions.

As noted above, the PDGF/VEGF family members act primarily by binding to receptor tyrosine kinases. In general, receptor tyrosine kinases are glycoproteins, which consist of an extracellular domain capable of binding a specific growth factor(s), a transmembrane domain, which is usually an alpha-helical portion of the protein, a juxtamembrane domain, which is where the receptor may be regulated by, e.g., protein phosphorylation, a tyrosine kinase domain, which is the enzymatic component of the receptor and a carboxy-terminal tail, which in many receptors is involved in recognition and binding of the substrates for the tyrosine kinase.

Five endothelial cell-specific receptor tyrosine kinases have been identified, belonging to two distinct subclasses: three vascular endothelial cell growth factor receptors, VEGFR-1 (Flt-1), VEGFR-2 (KDR/Flk-1), VEGFR-3 (Flt4), and the two receptors of the Tie family, Tie and Tie-2 (Tek). These receptors differ in their specificity and affinity. All of them have the intrinsic tyrosine kinase activity which is necessary for signal transduction.

The only receptor tyrosine kinases known to bind VEGFs are VEGFR-1, VEGFR-2 and VEGFR-3. VEGFR-1 and VEGFR-2 bind VEGF with high affinity, and VEGFR-1 also binds PlGF. VEGF-B binds to VEGFR-1 with high affinity, but not to VEGFR-2 or -3 (Olofsson et al, Proc. Natl. Acad. Sci. USA, 95:11709-11714 (1998)). VEGF-C has been shown to be the ligand for VEGFR-3, and it also activates VEGFR-2 (Joukov et al, The EMBO Journal 15:290-298 (1996)). VEGF-D binds to both VEGFR-2 and VEGFR-3 (Achen et al, Proc. Natl. Acad. Sci. USA 95:548-553 (1998)). A ligand for Tek/Tie-2 has been described in International Patent Application No. PCT/US95/12935 (WO 96/11269) by Regeneron Pharmaceuticals, Inc. The ligand for Tie has not yet been identified.

A novel 130-135 kDa VEGF isoform specific receptor also has been purified and cloned (Soker et al, Cell 92:735-745 (1998)). The VEGF receptor was found to specifically bind the VEGF₁₆₅ isoform via the exon 7 encoded sequence, which shows weak affinity for heparin (Soker et al, Cell 92:735-745 (1998)). Surprisingly, the receptor was shown to be identical to human neuropilin-1 (NP-1), a receptor involved in early stage neuromorphogenesis. PlGF-2 also appears to interact with NP-1 (Migdal et al, J. Biol. Chem. 273:22272-22278 (1998)).

VEGFR-1, VEGFR-2 and VEGFR-3 are expressed differently by endothelial cells. Generally, both VEGFR-1 and VEGFR-2 are expressed in blood vessel endothelia (Oelrichs et al, Oncogene 8:11-18 (1992); Kaipainen et al, J. Exp. Med. 178:2077-2088 (1993); Dumont et al, Dev. Dyn. 203:80-92 (1995); Fong et al, Dev. Dyn. 207:1-10 (1996)) and VEGFR-3 is mostly expressed in the lymphatic endothelium of adult tissues (Kaipainen et al, Proc. Natl. Acad. Sci. USA 9:3566-3570 (1995)). VEGFR-3 is also expressed in the blood vasculature surrounding tumors.

Although VEGFR-1 is mainly expressed in endothelial cells during development, it can also be found in hematopoetic precursor cells during early stages of embryogenesis (Fong et al, Nature 376:66-70 (1995)). In adults, monocytes and macrophages also express this receptor (Barleon et al, Blood 87:3336-3343 (1995)). In embryos, VEGFR-1 is expressed by most, if not all, vessels (Breier et al, Dev. Dyn. 204:228-239 (1995); Fong et al, Dev. Dyn. 207:1-10 (1996)).

Since the identification and characterization of VEGF, a number of important findings have focused attention on the activity of angiogenic factors and the elucidation of new factors. The early findings showed that angiogenesis is required for normal development and physiology. Processes such as embryogenesis, wound healing, and corpus luteum formation, all involve angiogenesis and angiogenic factors. During wound healing, for example, VEGF mRNA levels increase suggesting a direct correlation between the expression of VEGF and the healing process. Also, a defect in VEGF regulation might be associated with wound healing disorders (Frank, S., et al, J. Biol. Chem. 2705:12607-12613 (1995)).

Another important finding involves the connection between angiogenesis and tumor development. Both tumor growth and metastasis are angiogenesis-dependent processes (Folkman, J. and Shing, Y., J. Biol. Chem. 267: 10931-10934 (1992)). For example, when tumor cells are introduced into an animal, the expression pattern of VEGF mRNA reveals expression at the highest level in cells at the periphery of necrotic, tumor growth areas. Numerous blood vessels were identified within these areas. The expression of VEGF in these areas suggests that hypoxemia, a state of deficient oxygenation, triggers expression and release of VEGF in the necrotic tumor. The expression of VEGF-B also has been directly correlated with tumor growth (see U.S. Pat. No. 5,840,693). VEGF-B expression is especially up regulated in tumor-associated macrophages and also in ovarian epithelial tumors (Sowter et al, Lab Invest. 77:607-14, (1997)). VEGF-B mRNA can be detected in most tumor cell lines investigated, including adenocarcinoma, breast carcinoma, lymphoma, squamous cell carcinoma, melanoma, fibrosarcoma and Schwannoma (Salven et al, Am J Pathol. 153:103-8 (1998)).

It has been shown that members of the VEGF/PDGF family produce variant transcripts. VEGF has been shown to display different transcripts because of alternative splicing. The human VEGF gene has five different mRNA species (Neufeld et al, FASEB J. 13:9-22 (1999)), resulting in proteins differing in their molecular mass and biological properties (Carmeliet, P., Nat. Med. 6:389-395 (2000)). The hVEGF-A₁₆₅ isoform is the predominant transcript in most human tissues, giving rise to a polypeptide with affinity to the neuropilin-1 receptor, besides the binding to VEGFR1 and VEGFR2. The hVEGF₁₂₁ and hVEGF₁₈₉ isoforms are expressed in normal tissues at lower levels. The hVEGF₂₀₆ isoform is mainly expressed in embryonic tissues (Houck et al, Mol Endocrinol. 5:1806-14 (1991)), while hVEGF₁₄₅ can only be found in tumor cell lines (Poltorak et al, J Biol Chem. 272:7151-8 (1997)). Moreover, VEGF is also regulated in an isoform-specific way under pathological conditions. In lung and colon carcinomas, hVEGF₁₆₅ and hVEGF₁₂₁ are up-regulated, whereas hVEGF₁₈₉ is not changed, suggesting an isoform-specific role of VEGF in malignancy (Cheung et al, Hum Pathol. 29:910-4 (1998)). An isoform specific VEGF targeting experiment with murine VEGF-B has shown that mVEGF₁₆₄ and mVEGF₁₈₈ are more important for postnatal growth and maintenance of normal function of cardiovascular system, while mVEGF₁₂₀ initiates and promotes vasculogenesis (Carmeliet et al, Nat Med. 5:495-502 (1999)).

The placenta growth factor (PlGF) has three different isoforms, which are expressed in a tissue and development specific way (Maglione et al, Oncogene 8:925-31 (1993); Cao et al, Biochem Biophys Res Commun. 235:493-8 (1997)).

Two isoforms of VEGF-B, generated by alternative splicing of mRNA, have been recognized (Grimmond et al, Genome Res. 6:124-131 (1996); Olofsson et al, J. Biol. Chem. 271:19310-19317 (1996); Townson et al, Biochem. Biophys. Res. Commun. 220:922-928 (1996)). They are a cell associated form of 167 amino acid residues (VEGF-B₁₆₇) and a secreted form of 186 amino acid residues (VEGF-B₁₈₆). The isoforms have an identical N-terminal domain of 115 amino acid residues, excluding the signal sequence. The common N-terminal domain is encoded by exons 1-5. Differential use of the remaining exons 6A, 6B and 7 gives rise to the two splice isoforms. By the use of an alternative splice-acceptor site in exon 6, an insertion of 101 bp introduces a frame-shift and a stop of the coding region of VEGF-B₁₆₇ cDNA. Thus, the two VEGF-B isoforms have differing C-terminal domains.

The different C-terminal domains of the two splice isoforms of VEGF-B affect their biochemical and cell biological properties. The C-terminal domain of VEGF-B₁₆₇ is structurally related to the corresponding region in VEGF, with several conserved cysteine residues and stretches of basic amino acid residues. Thus, this domain is highly hydrophilic and basic and, accordingly, VEGF-B₁₆₇ will remain cell-associated on secretion, unless the producing cells are treated with heparin or high salt concentrations. The cell-associated molecules binding VEGF-B₁₆₇ are likely to be cell surface or pericellular heparin sulfate proteoglycans. It is likely that the cell-association of this isoform occurs via its unique basic C-terminal region.

The C-terminal domain of VEGF-B₁₈₆ has no significant similarity with known amino acid sequences in the databases. The hydrophobic character of the C-terminal domain of VEGF-B₁₈₆ contrasts with the properties of the hydrophilic and basic C-terminal domain of VEGF-B₁₆₇ This is supported by the observation that VEGF-B₁₈₆ does not remain cell-associated on its secretion. Recent evidence indicates that this isoform is proteolytically processed, which regulates the biological properties of the protein (Olofsson et al, Proc. Natl. Acad. Sci. USA, 95:11709-11714 (1998)).

A further difference is found in the glycosylation of the VEGF-B isoforms. VEGF-B₁₆₇ is not glycosylated at all, whereas VEGF-B₁₈₆ is O-glycosylated but not N-glycosylated.

Both isoforms of VEGF-B also form heterodimers with VEGF, consistent with the conservation of the eight cysteine residues involved in inter- and intramolecular disulfide bonding of PDGF-like proteins. Furthermore, co-expression of VEGF-B and VEGF in many tissues suggests that VEGF-B-VEGF heterodimers occur naturally. Heterodimers of VEGF-B₁₆₇-VEGF remain cell-associated. In contrast, heterodimers of VEGF-B₁₈₆ and VEGF are freely secreted from cells in a culture medium. VEGF also forms heterodimers with PlGF (DiSalvo, et al, J. Biol. Chem. 270:7717-7723 (1995)). The production of heterodimeric complexes between the members of this family of growth factors could provide a basis for a diverse array of angiogenic or regulatory molecules.

Since the secreted VEGF-B₁₆₇ remains cell-associated, it is intrinsically difficult to obtain significant amounts of soluble VEGF-B₁₆₇. Accordingly, there is a need to develop methods for increasing the amount of soluble VEGF-B₁₆₇.

### SUMMARY OF THE INVENTION

This invention relates to a N-glycosylated VEGF-B and a method for increasing the amount of soluble VEGF-B proteins.

In a first aspect, the invention provides a purified and isolated nucleic acid molecule having a polynucleotide sequence selected from the group consisting of SEQ ID NO:1 (sequence encoding VEGF-B₁₆₇), SEQ ID NO:3 (sequence encoding VEGF-B₁₈₆) and SEQ ID NO:5 (sequence encoding VEGF-B_{Ex1-5}) into which a nucleotide sequence encoding at least one putative N-glycosylation site has been inserted. The nucleic acid molecule having said polynucleotide sequence can be naked and/or in a vector or liposome. The putative N-glycosylation site is -NXT-, -NXS- or -NXC-, where N represents the amino acid asparagine, X may be any amino acid, and T, S and C represent the amino acids threonine, serine and cysteine, respectively. The nucleotide sequence which encodes the N-glycosylation site may thus comprise aay-nnn¹-(wgy/wcn)-nnn², with the proviso that -nnn¹- is not tga, tar or cnn, and -nnn²- is preferably not ccn, where w represents adenine or thymine/uracil, g represents guanine, y represents cytosine or thymine/uracil, c represents cytosine, n represents adenine, cytosine, guanine or thymine/uracil; t represents thymine/uracil, a represents adenine, and r represents guanine or adenine. (Rules for N-glycosylation are described at http://www.expasy.ch/cgi-bin/nicedoc.pl?PDOC00001). Preferably the nucleotide sequence comprises aay-nnn¹-(agy/wcn)-nnn².

The invention includes the nucleic acid molecules described above as well as fragments of those polynucleotides, and variants of those polynucleotides with sufficient similarity to the non-coding strand of those polynucleotides to hybridize thereto under stringent conditions and which can code for VEGF-B or a fragment or analog thereof which exhibits at least 90% sequence identity to SEQ ID NO:1, SEQ ID NO:3 or SEQ ID NO:5 and which binds to VEGFR-1. Thus, such polynucleotide fragments and variants having a nucleotide sequence encoding at least one putative N-glycosylation site inserted therein are intended as aspects of the invention. Exemplary stringent hybridization conditions are as follows: hybridization at 42° C. in 5×SSC, 20 mM NaPO₄, pH 6.8, 50% formamide; and washing at 42° C. in 0.2×SSC. Those skilled in the art understand that it is desirable to vary these conditions empirically based on the length and the GC nucleotide base content of the sequences to be hybridized, and that well accepted formulas for determining such variation exist. See for example Sambrook et al, “Molecular Cloning: A Laboratory Manual”, Second Edition, pages 9.47-9.51, Cold Spring Harbor, N.Y.: Cold Spring Harbor Laboratory (1989).

Moreover, purified and isolated nucleic acid molecules having a polynucleotide sequence encoding other, non-human, mammalian VEGF-B forms and having a nucleotide sequence encoding at least one putative N-glycosylation site inserted therein are aspects of the invention, as are the polypeptides encoded thereby.

A second aspect of the invention involves the purification and isolation of a protein having an amino acid sequence selected from the group consisting of SEQ ID NO:2 (VEGF-B₁₆₇), SEQ ID NO:4 (VEGF-B₁₈₆) and SEQ ID NO:6 (VEGF-B_{Ex1-5}) and having at least one putative N-glycosylation site inserted therein. The purified and isolated protein preferably is produced by the expression of the nucleic acid molecule of the invention. As noted above, the at least one putative N-glycosylation site is -NXT-, -NXS- or NXC, where N represents the amino acid asparagine, X may be any amino acid, and T, S and C represent the amino acids threonine, serine and cysteine, respectively. Preferably the N-glycosylation site is -NXT- or -NXS-, especially preferably -NXT-. It is also preferred that X and the amino acid following T or S not be proline.

As used herein, the term “VEGF-B” collectively refers to the known VEGF-B167 and VEGF-B186 polypeptide isoforms as well as to fragments or analogs thereof which exhibit at least 90% sequence identity to SEQ ID NO:1, SEQ ID NO:3 or SEQ ID NO:5 and which bind to VEGFR-1 and/or have the vasculogenesis stimulating activity of VEGF-B. The active substance preferably will include the amino acid sequence Pro-Xaa-Cys-Val-Xaa-Xaa-Xaa-Arg-Cys-Xaa-Gly-Cys-Cys (where Xaa may be any amino acid) which is characteristic of VEGF-B.

Polypeptides comprising conservative substitutions, insertions, or deletions, but which still retain the biological activity of VEGF-B are clearly to be understood to be within the scope of the invention. Persons skilled in the art will be well aware of methods which can readily be used to generate such polypeptides, for example the use of site-directed mutagenesis, or specific enzymatic cleavage and ligation. The skilled person will also be aware that peptidomimetic compounds or compounds in which one or more amino acid residues are replaced by a non-naturally occurring amino acid or an amino acid analog may retain the required aspects of the biological activity of VEGF-B. Such compounds can readily be made and tested by methods known in the art, and are also within the scope of the invention.

In addition, possible variant forms of the VEGF-B polypeptide which may result from alternative splicing, as are known to occur with VEGF and VEGF-B, and naturally-occurring allelic variants of the nucleic acid sequence encoding VEGF-B are encompassed within the scope of the invention. Allelic variants are well known in the art, and represent alternative forms or a nucleic acid sequence which comprise substitution, deletion or addition of one or more nucleotides, but which do not result in any substantial functional alteration of the encoded polypeptide.

Such variant forms of VEGF-B can be prepared by targeting non-essential regions of the VEGF-B polypeptide for modification. These non-essential regions are expected to fall outside the strongly-conserved regions of the VEGF/PDGF family of growth factors. In particular, the growth factors of the VEGF family, including VEGF-B, are dimeric, and VEGF, VEGF-B, VEGF-C, VEGF-D, PlGF, PDGF-A and PDGF-B show complete conservation of eight cysteine residues in the N-terminal domains, i.e. the PDGF/VEGF-homology domains (Olofsson et al., Proc. Natl. Acad. Sci. USA, 1996 93 2576-2581; Joukov et al., EMBO J., 1996 15 290-298). These cysteines are thought to be involved in intra- and inter-molecular disulfide bonding. In addition there are further strongly, but not completely, conserved cysteine residues in the C-terminal domains. Loops 1, 2 and 3 of each subunit, which are formed by intra-molecular disulfide bonding, are involved in binding to the receptors for the PDGF/VEGF family of growth factors (Andersson et al., Growth Factors, 1995 12 159-164).

Persons skilled in the art thus are well aware that in most cases these cysteine residues should be preserved in any proposed variant form, although there may be exceptions since receptor-binding VEGF-B analogs are known in which one or more of the cysteines is not conserved. Similarly, a skilled worker would be aware that the active sites present in loops 1, 2, and 3 also should be preserved. However, other regions of the molecule can be expected to be of lesser importance for biological function, and therefore offer suitable targets for modification. Modified polypeptides can readily be tested for their ability to show the biological activity of VEGF-B by routine activity assay procedures such as the endothelial cell proliferation assay.

Preferably where amino acid substitution is used, the substitution is conservative, i.e. an amino acid is replaced by one of similar size and with similar charge properties. As used herein, the term “conservative substitution” denotes the replacement of an amino acid residue by another, biologically similar residue, i.e., one that has similar properties. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids which can be substituted for one another include asparagine, glutamine, serine and threonine. The term “conservative substitution” also includes the use of a substituted amino acid in place of an unsubstituted parent amino acid. Exemplary conservative substitutions are set out in the following Table A:

TABLE A
Conservative Substitutions I

| SIDE CHAIN | |
|---|---|
| CHARACTERISTICS | AMINO ACID |
| Aliphatic | |
| Non-polar | G A P |
| | I L V |
| Polar-uncharged | C S T M |
| | N Q |
| Polar-charged | D E |
| | K R |
| Aromatic | H F W Y |
| Other | N Q D E |

Alternatively, conservative amino acids can be grouped as described in Lehninger, [Biochemistry, Second Edition; Worth Publishers, Inc. New York, N.Y. (1975), pp.71-77] as set out in the following Table B.

TABLE B
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Exemplary conservative substitutions also are set out in the following Table C.

TABLE C
Conservative Substitutions III

| Original | Exemplary |
|---|---|
| Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, |
| | Ala, Phe, |
| Leu (L) | Ile, Val, Met, |
| | Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, |
| | Phe, Ala |

If desired, the VEGF-B proteins of the invention can be modified, for instance, by amidation, carboxylation, or phosphorylation, or by the creation of acid addition salts, amides, esters, in particular C-terminal esters, and N-acyl derivatives of the peptides of the invention. The proteins also can be modified to create peptide derivatives by forming covalent or noncovalent complexes with other moieties. Covalently-bound complexes can be prepared by linking the chemical moieties to functional groups on the side chains of amino acids comprising the peptides, or at the N- or C-terminus.

In particular, it is anticipated that the VEGF-B proteins can be conjugated to a reporter group, including, but not limited to a radiolabel, a fluorescent label, an enzyme (e.g., that catalyzes a colorimetric or fluorometric reaction), a substrate, a solid matrix, or a carrier (e.g., biotin or avidin). The polypeptide can be linked to an epitope tag, such as the FLAG® octapeptide (Sigma, St. Louis, Mo.) or histidine, to assist in affinity purification. Also the polypeptides according to the invention may be labeled with a detectable label. The polypeptide may be covalently or non-covalently coupled to a suitable supermagnetic, paramagnetic, electron dense, ecogenic or radioactive agent for imaging. For use in diagnostic assays, radioactive or non-radioactive labels may be used. Examples of radioactive labels include a radioactive atom or group, such as ¹²⁵I or ³²p. Examples of non-radioactive labels include enzymatic labels, such as horseradish peroxidase or fluorimetric labels, such as fluorescein-5-isothiocyanate (FITC). Labeling may be direct or indirect, covalent or non-covalent.

The modified polypeptides can readily be tested for their ability to show the biological activity of VEGF-B by routine activity assay procedures such as the fibroblast proliferation assay.

It will be clearly understood that nucleic acids and polypeptides of the invention may be prepared by synthetic means or by recombinant means, or may be purified from natural sources.

As used herein, the term “comprising” means “included but not limited to”. The corresponding meaning applies to the word “comprises”.

A third aspect of the invention provides vectors comprising the nucleic acid molecule of the first aspect of the invention, and host cells transformed or transfected with nucleic acids molecules or vectors of the invention. These may be eukaryotic or prokaryotic in origin. These cells are particularly suitable for expression of the polypeptide of the invention, and include insect cells such as Sf9 or HF cells, obtainable from the American Type Culture Collection, infected with a recombinant baculovirus, and the human embryo kidney cell line 293-EBNA transfected by a suitable expression plasmid. Preferred vectors of the invention are expression vectors in which a nucleic acid according to the invention is operatively connected to one or more appropriate promoters and/or other control sequences, such that appropriate host cells transformed or transfected with the vectors are capable of expressing the polypeptide of the invention. Other preferred vectors are those suitable for transfection of mammalian cells, or for gene therapy, such as adenoviral-, vaccinia- or retroviral-based vectors or liposomes. A variety of such vectors are known in the art.

The invention also provides a method of making a vector capable of expressing a polypeptide encoded by a nucleic acid according to the invention, comprising the steps of operatively connecting the nucleic acid molecule of the first aspect to one or more appropriate promoters and/or other control sequences, as described above.

The invention further provides a method of making a polypeptide according to the invention, comprising the steps of expressing a nucleic acid or vector of the invention in a host cell, and isolating the polypeptide from the host cell or from the host cell's growth medium.

The polypeptide according to the invention may be employed in combination with a suitable pharmaceutical carrier. The resulting compositions comprise an effective amount of glycosylated VEGF-B or a pharmaceutically acceptable non-toxic salt thereof, and a pharmaceutically acceptable solid or liquid carrier or adjuvant. Examples of such a carrier or adjuvant include, but are not limited to, saline, buffered saline, Ringer's solution, mineral oil, talc, corn starch, gelatin, lactose, sucrose, microcrystalline cellulose, kaolin, mannitol, dicalcium phosphate, sodium chloride, alginic acid, dextrose, water, glycerol, ethanol, thickeners, stabilizers, suspending agents and combinations thereof. Such compositions may be in the form of solutions, suspensions, tablets, capsules, creams, salves, elixirs, syrups, wafers, ointments or other conventional forms. The formulation to suit the mode of administration. Compositions can comprise a glycosylated VEGF-B and optionally further comprise one or more of PDGF-A, PDGF-B, VEGF, non-glycosylated VEGF-B, VEGF-C, VEGF-D, PlGF and/or heparin. Compositions comprising the glycosylated VEGF-B will contain from about 0.1% to 90% by weight of the active compound(s), and most generally from about 10% to 30%.

For intramuscular preparations, a sterile formulation, preferably a suitable soluble salt form of the glycosylated VEGF-B, such as hydrochloride salt, can be dissolved and administered in a pharmaceutical diluent such as pyrogen-free water (distilled), physiological saline or 5% glucose solution. A suitable insoluble form of the compound may be prepared and administered as a suspension in an aqueous base or a pharmaceutically acceptable oil base, e.g. an ester of a long chain fatty acid such as ethyl oleate.

In a further aspect, the invention provides a method for making a soluble VEGF-B₁₆₇ from a host cell and a method for increasing an amount of a soluble VEGF-B₁₆₇₁ VEGF-B₁₈₆ or VEGF-B_{Ex1-5} protein from a host cell. These methods comprise inserting at least one putative N-glycosylation site into a nucleotide sequence which codes for VEGF-B₁₆₇, VEGF-B₁₈₆ or VEGF-BE_{EX1-5} protein; transforming or transfecting said nucleotide sequence with the inserted N-glycosylation site into a host cell; culturing the transfected host cell in a growth medium such that said nucleotide sequence with inserted N-glycosylation site is expressed; and isolating the expressed polypeptide from the growth medium in which said host cell was cultured. These methods can further comprise exposing the cultured transfected host cell to heparin after said polypeptide is expressed.

### BRIEF DESCRIPTION OF THE DRAWINGS

The invention will be described in further detail hereinafter with reference to the accompanying drawings in which:

FIG. 1 is an alignment of the amino acid sequences of the VEGF homology domain (VHD) of VEGF-A and PlGF with VEGF-B;

FIG. 2 is a diagram of plasmid pSecTagA-hVEGF-B₁₈₆-H₆-NXT containing a nucleotide sequence encoding VEGF-B₁₈₆ having an N-glycosylation site incorporated therein;

FIG. 3 is a diagram of plasmid pSecTagA-hVEGF-B₁₆₇-H₆-NXT containing a nucleotide sequence encoding VEGF-B₁₆₇ having an N-glycosylation site incorporated therein;

FIG. 4 is a diagram of plasmid pSecTagA-hVEGF-B-Exon1-5-H₆-NXT containing a nucleotide sequence encoding exons 1-5 of VEGF-B having an N-glycosylation site incorporated therein;

FIG. 5 shows the expression of hVEGF-B₁₆₇ with and without the potential glycosylation site (NXT);

FIG. 6 shows the expression of hVEGF-B₁₆₇ and hVEGF-B₁₈₆ with and without the potential glycosylation site (NXT);

FIG. 7 shows the expression and receptor binding of hVEGF-B₁₆₇ and hVEGF-B₁₈₆ with and without the potential glycosylation site (NXT); and

FIG. 8 shows the expression and receptor binding of polypeptide encoded by exons 1-5 of hVEGF-B with and without the potential glycosylation site (NXT).

### DETAILED DESCRIPTION OF PREFERRED EMBODIMENTS

### EXAMPLE 1

Introduction of the Glycosylation Site

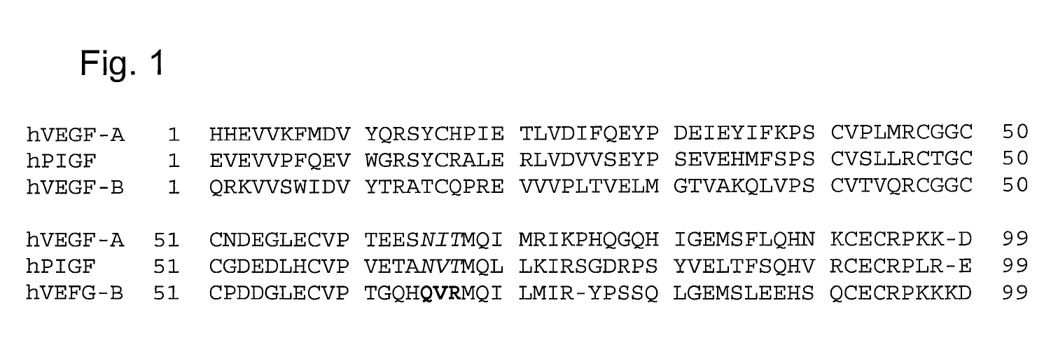

As mentioned before, VEGF-B is a PDGF/VEGF family member that is completely devoid of any N-glycosylation. To analyze the effects of N-glycosylation on VEGF-B, a N-glycosylation site was introduced into VEGF-B. To determine the most appropriate site to introduce a mutation that would lead to N-glycosylation of VEGF-B, the amino acid sequences of the first 99 amino acids of VEGF-A, PlGF and VEGF-B, respectively, were aligned (see FIG. 1). The N-glycosylation sites of VEGF-A and PlGF at amino acids 65-67 are italicized in FIG. 1. Nucleotides encoding a putative N-glycosylation site (NXT) were inserted at the position corresponding to nucleotides 286-294 of hVEGF-B (SEQ ID NO:1). The replaced nucleotides normally found at positions 286-294 encode the amino acid residues QVR and these amino acid residues are bolded in FIG. 1.

### EXAMPLE 2

Preparation of Recombinant Vectors

Six mammalian expression vectors for both naturally occurring isoforms of VEGF-B (i.e., VEGF-B₁₆₇ and VEGF-B₁₈₆) and for an artificial splice variant (comprising exons 1 to 5 only) were constructed with and without the putative N-glycosylation site.

Using PCR, nucleotides coding for a histidine tag were added to the C-terminal end of a nucleotide sequence coding for hVEGF-B₁₈₆. A nucleotide sequence coding for hVEGF-B₁₈₆-H₆ was then inserted into pSecTagA (Invitrogen, Carlsbad, Calif.) using standard cloning procedures to construct pSecTagA-hVEGF-B₁₈₆-H₆. The full sequence of pSecTagA-hVEGF-B₁₈₆-H₆ is given in SEQ ID NO:7.

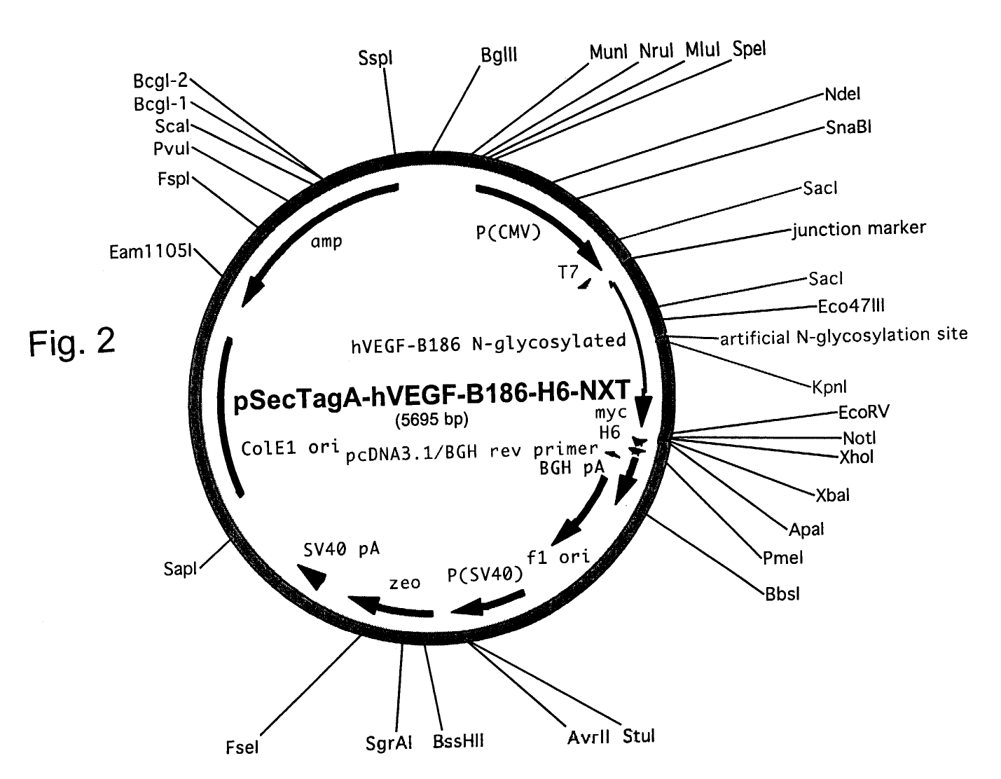

To construct pSecTagA-hVEGF-B₁₈₆-H₆-NXT, a PCR product of covering nucleotides 1-325 from Genebank Acc. No. U48801 was produced which introduced a N-glycosylation site at nucleotide positions 289-297 using the 3′ primer: 5′-TCGGTACCGGATCATGAGGATCTGCATGGTGACGTTGTGCTGCCCAGTGGCCA-3′ (SEQ ID NO:8). This PCR product was then cloned into a plasmid with full-length hVEGF-B₁₈₆ where it used to replace the corresponding sequence to produce hVEGF-B₁₈₆-NXT. A histidine tag was then added by cloning together the N-terminal portion of hVEGF-B₁₈₆-NXT with the C-terminal portion of hVEGF-B₁₈₆-H₆ using standard cloning procedures to produce hVEGF-B₁₈₆-H₆-NXT. The nucleotide sequence coding for hVEGF-B₁₈₆-H₆-NXT was then inserted into pSecTagA (Invitrogen) using standard cloning procedures to construct pSecTagA-hVEGF-B₁₈₆-H₆-NXT. The full sequence of pSecTagA-hVEGF-B₁₈₆-H₆-NXT is given in SEQ ID NO:9, and the plasmid is illustrated in FIG. 2.

To construct pSecTagA-hVEGF-B₁₆₇-H₆, a 349 bp PCR product was produced covering nucleotides 250-567 from Genebank Acc. No. U48801, nucleotides coding for the histidine tag, a stop codon, the NotI restriction site and terminal clamp nucleotides using the 5′ primer: 5′-CCTGACGATGGCCTGGAGTGT-3′ (SEQ ID NO:10) and the 3′ primer: 5′-GAGCGGCCGCTCAATGATGATGATGATGATGCCTTCGCAGCTTCCGGCAC-3′ (SEQ ID NO:11) and hVEGF-B₁₆₇ as the template. The 349 bp PCR product was cut with KpnI and NotI and the KpnI-NotI fragment was inserted into pSecTagA-hVEGF-B₁₈₆-H₆ to replace the KpnI-NotI fragment removed from this vector using standard cloning procedures. The full sequence of pSecTagA-hVEGF-B₁₆₇-H₆ is given in SEQ ID NO:12.

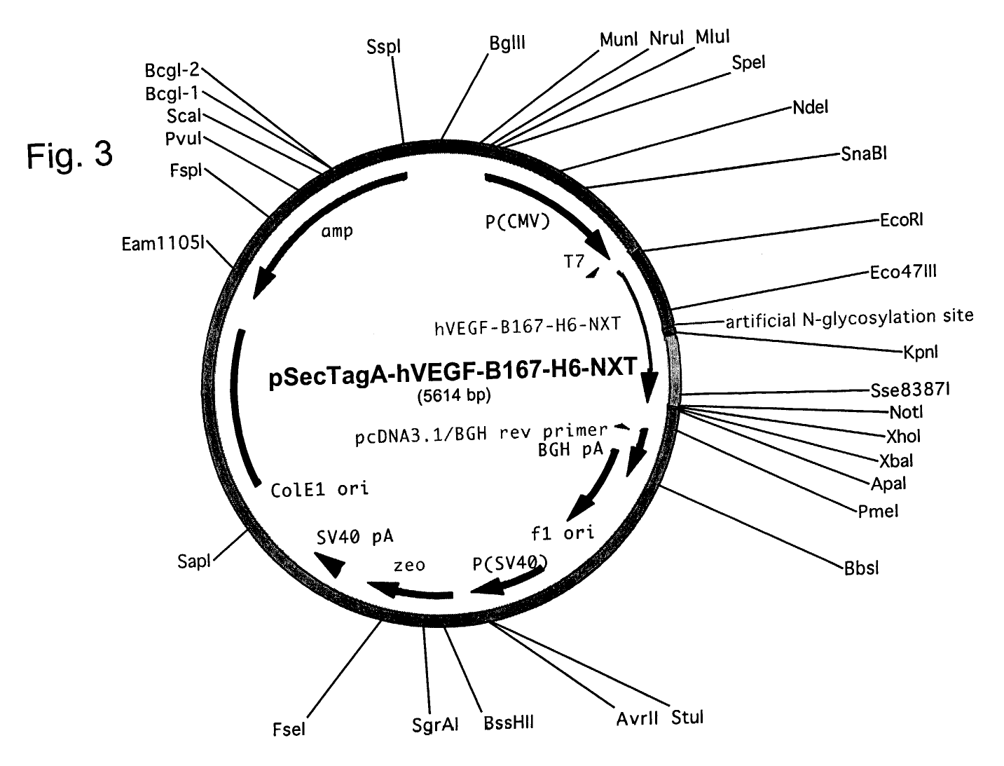

Similarly, pSecTagA-hVEGF-B₁₆₇-H₆-NXT was constructed as above except the KpnI-NotI fragment was inserted into pSecTagA-hVEGF-B₁₈₆-H₆-NXT to replace the KpnI-NotI fragment removed from this vector. The full sequence of pSecTagA-hVEGF-B₁₆₇-H₆-NXT is given in SEQ ID NO:13, and the plasmid is illustrated in FIG. 3.

To construct pSecTagA-hVEGF-B_{Ex1-5}-H₆, a 443 bp PCR product was obtained covering nucleotides 1-411 from Genebank Acc. No. U48801, nucleotides coding for the histidine tag, a stop codon, the NotI restriction site and terminal clamp nucleotides using the 5′ primer: 5′-CACCATGAGCCCTCTGCTCC-3′ (SEQ ID NO:14) and 3′ primer: 5-GAGCGGCCGCTCAGTGGTGATGATGATGGTCTGGCTTCACAGCACTG-3′ (SEQ ID NO:15) and hVEGF-B₁₆₇ as the template. The PCR product was cut with KpnI and NotI and the resulting 320 bp fragment was inserted into pSecTagA-hVEGF-B₁₈₆-H₆-NXT to replace the KpnI-NotI removed from this vector using standard cloning procedures. The full sequence of pSecTagA-hVEGF-B_{Ex1-5}-H₆ is given in SEQ ID NO:16.

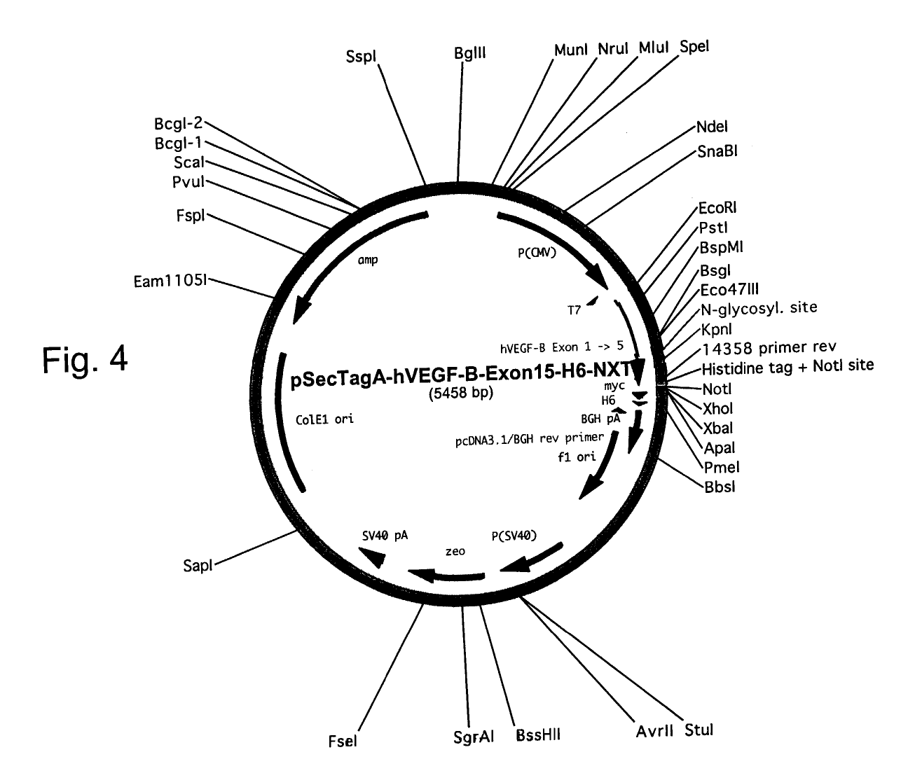

To construct pSecTagA-hVEGF-B_{Ex1-5}-H₆-NXT, the same procedures as above were used except the KpnI-NotI fragment was inserted into pSecTagA-hVEGF-B₁₈₆-H₆-NXT to replace the KpnI-NotI fragment removed from this vector. The full sequence of pSecTagA-hVEGF-B_{Ex1-5} -H₆-NXT is given in SEQ ID NO:17, and the plasmid is illustrated in FIG. 4.

The following Table D lists the expression vectors for the naturally occurring 186 and 167 amino acid isoforms of VEGF-B and for the artificial splice variant (comprising exon 1 to 5 only), constructed with and without the potential glycosylation site (NXT).

TABLE D

| Construct Name | Protein |
|---|---|
| pSecTagA-hVEGF-B₁₈₆-H₆ | histidine-tagged VEGF-B₁₈₆ |
| psecTagA-hVEGF-B₁₈₆-H₆-NXT | histidine-tagged and |
| | N-glycosylated VEGF-B₁₈₆ |
| pSecTagA-hVEGF-B₁₆₇-H₆ | histidine-tagged VEGF-B₁₆₇ |
| pSecTagA-hVEGF-B₁₆₇-H₆-NXT | histidine-tagged and |
| | N-glycosylated VEGF-B₁₆₇ |
| pSecTagA-hVEGF-B-Exon1-5-H₆ | histidine-tagged VEGF-B |
| | Exons 1 to 5 only |
| pSecTagA-hVEGF-B-Exon1-5-H₆-NXT | histidine-tagged and |
| | N-glycosylated VEGF-B |
| | Exons 1 to 5 only |

### EXAMPLE 3

Transfection and Expression of Recombinant Proteins

The six mammalian expression vectors of human VEGF-B described above along with expression vectors containing histidine-tagged VEGF (positive control), a histidine-tagged VHD of VEGF-C (negative control) and histidine-tagged hybrid 84-11 (positive control), respectively, were transfected into 293T cells using CaPO₄-mediated transfection according to procedures described in Sambrook, J. et al., Molecular Cloning, A Laboratory Manual, (Cold Spring Harbor Press, Cold Spring Harbor, N.Y.), 16.33-16.36 (1989). 48 hours after transfection, the cells were metabolically labeled with S³⁵ methionine and S³⁵ cysteine (Promix, Amersham) for 12 to 24 hours. The conditioned supernatant was subjected to immunoprecipitation with an antiserum specific to human VEGF-B (874) and a monoclonal antibody specific to pentahistidine (H₅ mAb, Qiagen).

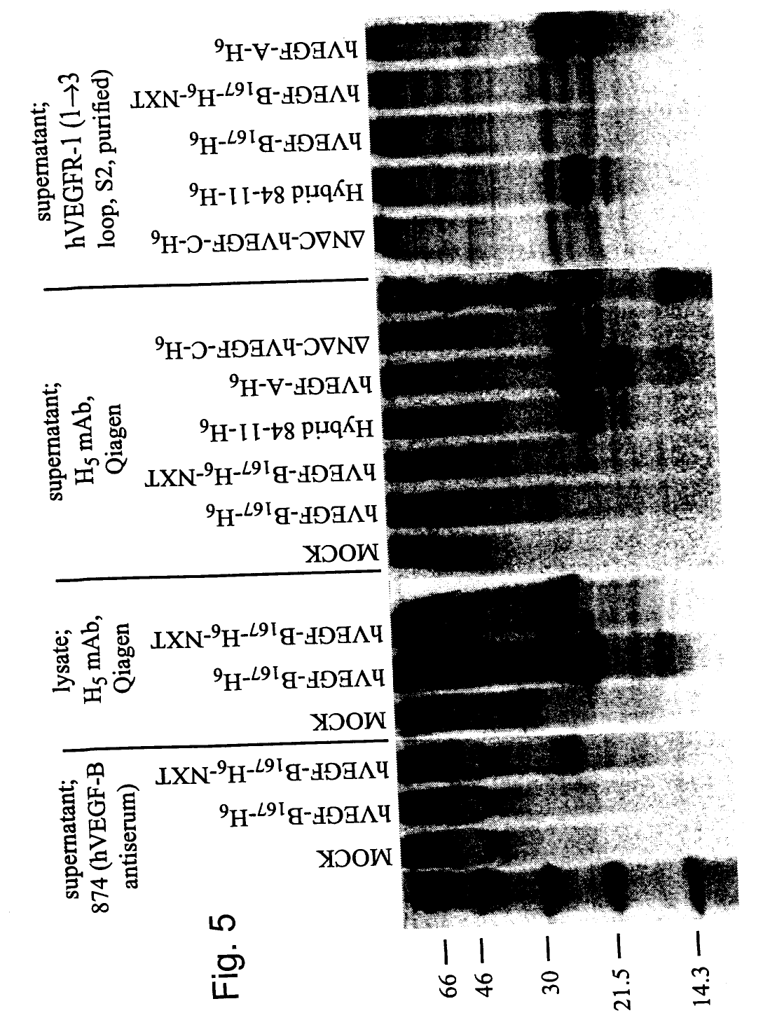

As seen in FIGS. 5 through 8, the three constructs produced with the inserted putative N-glycosylation site are glycosylated.

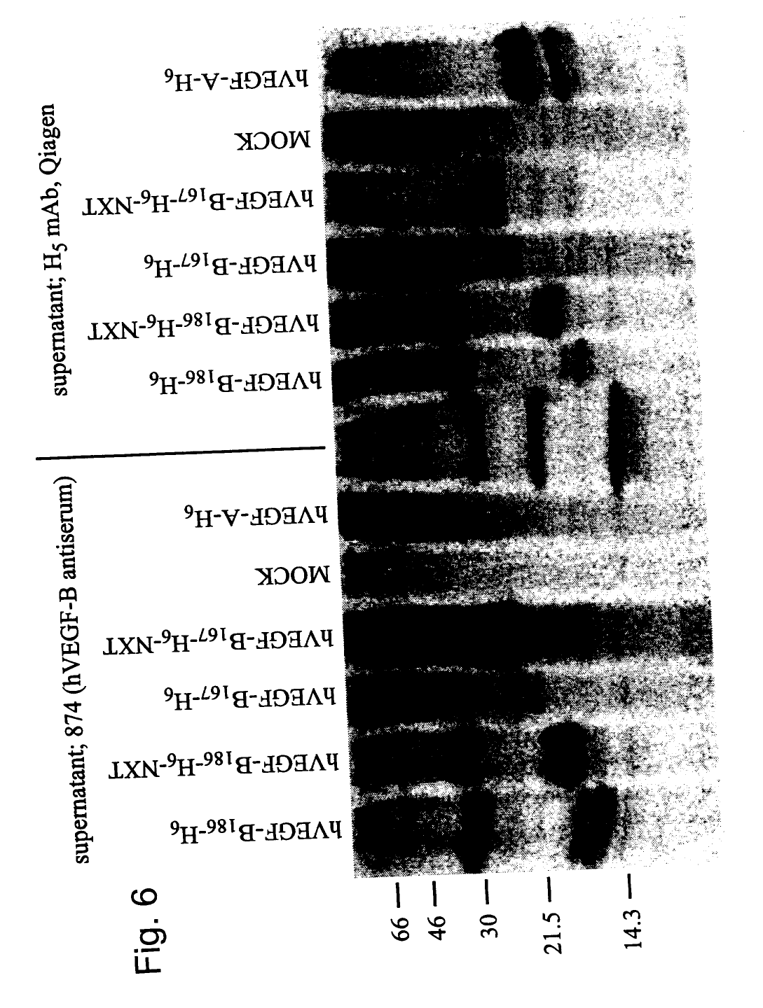

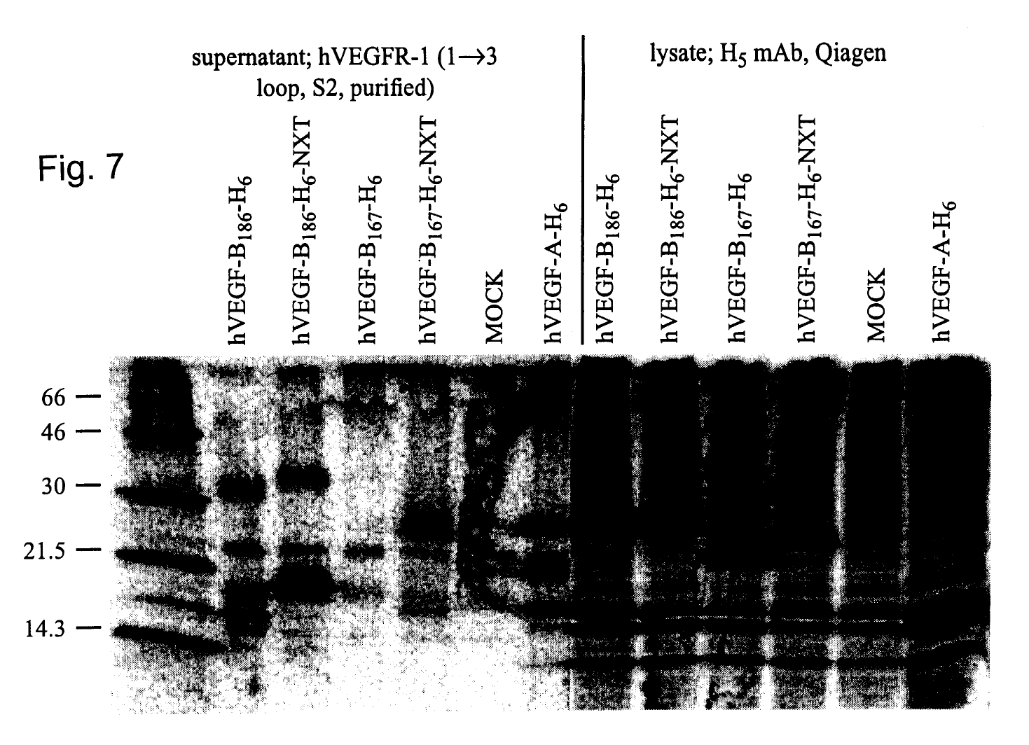

As can be seen from FIGS. 5-7, comparison of supernatants and lysates and using heparin to release cell bound proteins shows that VEGF-B₁₆₇ is almost completely retained at the cell surface or within the cell. About a 50 fold increase of VEGF-B₁₆₇ can be detected in the supernatant upon glycosylation (FIG. 5). As shown in FIGS. 6 and 7, VEGF-B₁₆₇ is released into the supernatant by treatment with 100 μg/ml heparin two hours prior to harvest. It was also found that approximately two times more glycosylated VEGF-B₁₆₇ can be detected in the supernatant of non-heparin treated 293T cells as compared to non-glycosylated VEGF-B₁₆₇ treated with 200 μg/ml heparin for two hours prior to harvesting. In addition, there is about a three fold increase in the amount of the glycosylated VEGF-B₁₆₇ detected in the supernatant by treatment with 200 μg/ml heparin two hours prior to harvest as compared to glycosylated VEGF-B₁₆₇ without heparin treatment, and approximately a six fold increase in the amount of the glycosylated VEGF-B₁₆₇ detected in the supernatant by treatment with 200 μg/ml heparin two hours prior to harvest as compared to the amount of non-glycosylated VEGF-B₁₆₇ detected in the supernatant with the same heparin treatment.

FIGS. 6 and 7 show that VEGF-B₁₈₆ is also partially retained at the cell surface and within the cell. In contrast to VEGF-B₁₆₇, almost all of the VEGF-B₁₈₆ is released into the supernatant upon glycosylation and heparin treatment (FIGS. 6 and 7). There seems to be no significant difference in the amount of VEGF-B₁₈₆ found in the supernatant between heparin-treated and untreated 293T cells. Thus the difference of VEGF-B₁₈₆ and N-glycosylated VEGF-B₁₈₆ protein levels in the supernatant (approximately three times more glycosylated VEGF-B₁₈₆) seems to be mainly due to enhanced secretion and/or production and not due to the release of cell surface bound protein.

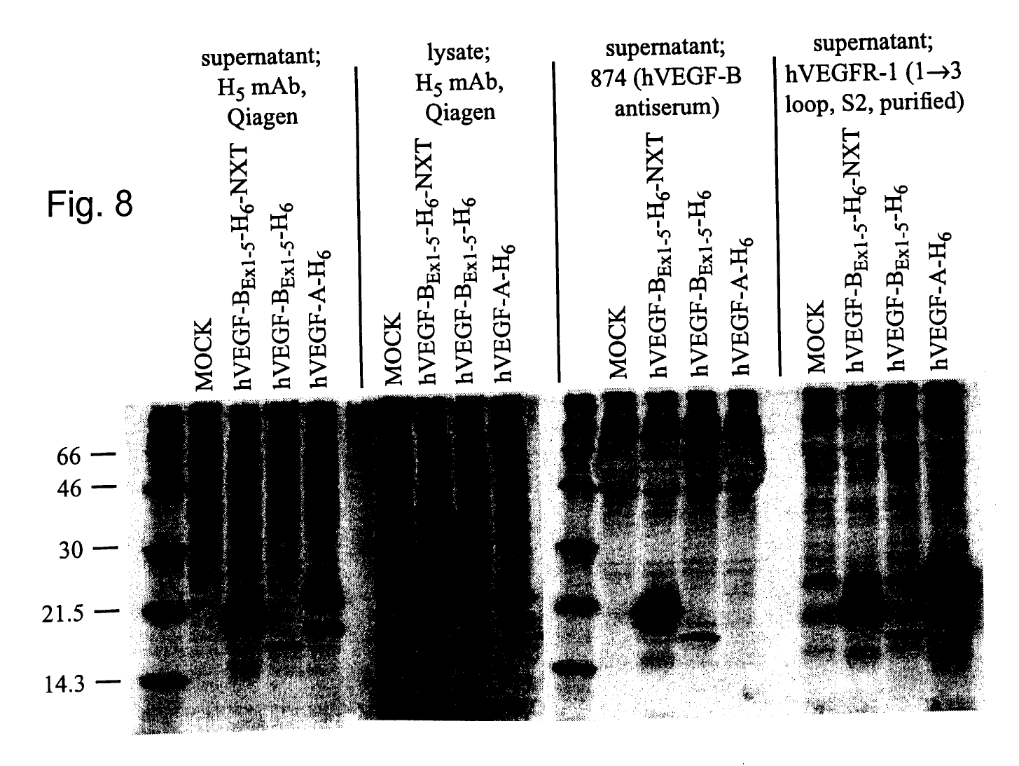

FIG. 8 shows that VEGF-B_{Exon1-5} is only efficiently released into the medium if it is N-glycosylated (over a 50 fold increase in soluble protein). This is unexpected since the signals retaining VEGF-B at the cell surface are thought to reside in the exon 6 and 7 encoded domains (FIG. 8). Treatment with heparin was not determined for this same reason.

### EXAMPLE 4

VEGF Receptor 1 Binding of Recombinant Proteins

The ability of the recombinant VEGF-B to bind VEGF receptor 1 (VEGFR-1) was analyzed using soluble fusion proteins consisting of the extracellular domain of VEGFR-1 and the Fc portion of human IgG1 (VEGFR-1-Fc). Biosynthetically labeled conditioned medium derived from 293T cells transfected as above in Example 3 were immunoprecipitated with protein A sepharose (PAS) bound to the VEGFR-1-Ig. Beads were washed three times with PBS, the bound protein eluted and resolved by reducing SDS-PAGE (15%). The dried gels were exposed to phosphoimager plates for 12-24 hours. Additionally, the cell lysates were immunoprecipitated with H₅ mAb.

When significant amounts of VEGF-B were present in the supernatant, binding to VEGFR-1 could be observed. This was seen with VEGF-B₁₈₆-H₆ after treatment with 100 μg/ml heparin two hours prior to harvest, VEGF-B₁₈₆-NXT-H₆ and VEGF-B Exon 1-5-NXT-H₆ (FIGS. 7 and 8).

### EXAMPLE 5

Stimulation of BaF3 VEGFR-01EC/EpoR Cell Survival

The effects of introducing the N-glycosylation site into VEGF-B can be assayed by measuring the ability of conditioned media from cells transfected with VEGF-B167 and VEGF-B167-NXT and/or VEGF-B186 and VEGF-B186-NXT to stimulate the survival of BaF3 VEGFR-01EC/EpoR cells. For the assay, BaF3 cells are used that are stably transfectd with a chimeric receptor consisting of the extracellular domain of VEGF receptor 1 and the intracellular domain of the erythropoietin receptor. For survival, these cells need either interlukin-3 or any growth factor capable of binding VEGFR-1, e.g., VEGF-A, VEGF-B or PlGF. Cells are plated to 96-well plates at a density of 20,000/well and grown in the presence of different amounts of medium conditioned by 293T cells that have been transfected with VEGF-B167 and VEGF-B167-NXT, VEGF-B186 and VEGF-B186-NXT, or both. Conditioned medium from 293T cells transfected with a mock (i.e., empty) vector may be used as a control. Prior to the assay, the conditioned medium should be cleared from potentially interfering proteins by immunoprecipitation using appropriate antibodies. For example, VEGF-A may be cleared from the conditioned medium prior to the assay using a mixture of monoclonal and polyclonal anti-hVEGF antibodies, commercially available from R&D Systems, Minneapolis, Minn. It is not necessary to preclear medium of PlGF as the amounts expressed by COS cells (if any) are negligible and its effects are not visible in the baseline noise. After 48 hours, an MTT (3-[4,5-dimethylthiazol-2-yl]-2,5-diphenyl-tetrazolium bromide thiazole blue) colorimetric assay may be performed and data collected at 540 nm using a microtitreplate reader.

To create the BglII site in the coding sequence of human VEGFR-1 just before the transmembrane domain, basepairs 1998-2268 of VEGFR-1 were PCR amplified with primers 5′-CCTCAGTGATCACACAGTGG-3′, containing the endogenous BclI site, and 5′-CAGAGATCTATTAGACTTGTCC-3′, containing a BglII site, and the PCR fragment was cloned into the BclI-BglII sites of VEGFR-1 in pBlueScript SKII+ (Stratagene) vector. The transmembrane and intracellular domains of the human erythropoietin receptor were excised from EpoR×B+B/pcDNAI and subcloned into the resulting plasmid as a BglII/NotI fragment. The EpoR×B+B is a clone of EpoR which has an internal BglII site added at the putative transmembrane (TM)/extracellular (EC) domain junction for the in-frame ligation of RTK extracellular domains. The vector backbone is pCDNA1-amp (˜5.4 kb, the original 1.75 kb EpoR clone was subcloned into pCDNA1-amp using KpnI, the sequence was reported by the Lodish Laboratory, MIT). An ˜1 kb fragment can be excised from this clone using BglII (5′)- NotI (3′) digest which contains the TM and cytoplasmic domain of EpoR.

The VEGFR-1/EpoR construct was finally subcloned into the pEF-BOS vector (Mizushima et al., Nucleic Acids Research, 18(17):5322 Sep. 11, 1990) as a KpnI/NotI fragment. The resulting plasmid was electroporated into BaF3 cells and stable cell pools were generated by selection with 250 micrograms/mL zoecin.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include everything within the scope of the appended claims and equivalents thereof.

What is claimed is:

1. An isolated nucleic acid molecule comprising:
a polynucleotide sequence selected from the group consisting of SEQ ID NO:1, SEQ ID NO:3, SEQ ID NO:5 or a polynucleotide sequence which hybridizes under stringent conditions with at least one of the foregoing sequences; and
a nucleotide sequence encoding at least one putative N-glycosylation site inserted therein.

2. An isolated polypeptide having an amino acid sequence selected from the group consisting of SEQ ID NO:2, SEQ ID NO:4 and SEQ ID NO:6 and having at least one putative N-glycosylation site inserted therein.

3. The isolated nucleic acid molecule of claim 1, wherein the at least one putative N-glycosylation site consists of a nucleotide sequence that encodes an amino acid sequence of NXT.

4. The isolated nucleic acid molecule of claim 1, wherein the at least one putative N-glycosylation site is inserted at nucleotides 286-294 of SEQ ID NO:1, SEQ ID NO:3 or SEQ ID NO:5.

5. An isolated polypeptide produced by expression of the nucleic acid molecule of claim 1.

6. An isolated polypeptide of claim 2 which binds a Vascular Endothelial Growth Factor Receptor-1.

7. A vector comprising a nucleic acid molecule of claim 1.

8. A host cell transformed or transfected with a vector according to claim 7.

9. A pharmaceutical composition comprising an effective amount of a polypeptide of claim 2.

10. The pharmaceutical composition of claim 9, further comprising heparin.

11. A method of making a soluble VEGF-B₁₆₇ from a host cell, comprising:
inserting at least one putative N-glycosylation site into a nucleotide sequence of SEQ ID NO:1;
transforming or transfecting said nucleotide sequence with inserted N-glycosylation site into a host cell;
culturing the transfected host cell in a growth medium such that said nucleotide sequence with inserted N-glycosylation site is expressed; and
isolating the expressed polypeptide from the growth medium in which said host cell was cultured.

12. The method of claim 11, further comprising exposing the cultured transfected host cell to heparin after said polypeptide is expressed.

13. The method of claim 11, wherein the at least one putative N-glycosylation site consists of a nucleotide sequence that encodes an amino acid sequence of NXT.

14. The method of claim 11, wherein the nucleotide sequence encoding the at least one putative N-glycosylation site is inserted at nucleotides 286-294 of SEQ ID NO:1.

15. A method of increasing an amount of a soluble VEGF-B₁₆₇, VEGF-B₁₈₆ or VEGF-B_{Ex1-5} polypeptide from a host cell, comprising:
inserting at least one putative N-glycosylation site into a nucleotide sequence selected from the group of nucleotides sequences of SEQ ID NO:1, SEQ ID NO:3 or SEQ ID NO:5;
transforming or transfecting said nucleotide sequence with inserted N-glycosylation site into a host cell;
culturing the transfected host cell in a growth medium such that said nucleotide sequence with inserted N-glycosylation site is expressed; and
isolating the expressed polypeptide from the growth medium in which said host cell was cultured.

16. The method of claim 15, further comprising exposing the cultured transfected host cell to heparin after said polypeptide is expressed.

17. The method of claim 15, wherein the at least one putative N-glycosylation site consists of a nucleotide sequence that encodes an amino acid sequence of NXT.

18. The method of claim 15, wherein the nucleotide sequence encoding the at least one putative N-glycosylation site is inserted at nucleotides 286-294 of SEQ ID NO:1, SEQ ID NO:3 or SEQ ID NO:5.
